(19) United States
(12) Patent Application Publication
(10) Pub. No.: US 2023/0014549 A1
(43) Pub. Date: Jan. 19, 2023

(54) MESENCHYMAL STEM CELLS FOR USE IN THE TREATMENT OF CHRONIC KIDNEY DISEASE

(71) Applicant: Boehringer Ingelheim Veterinary Medicine Belgium, Evergem (BE)

(72) Inventors: Jan SPAAS, Evergem (BE); Charlotte BEERTS, Evergem (BE); Eva DEPUYDT, Evergem (BE)

(21) Appl. No.: 17/857,804

(22) Filed: Jul. 5, 2022

(30) Foreign Application Priority Data
Jul. 8, 2021 (EP) EP21184504.5

(51) Int. Cl. A61K 35/28; A61P 13/12

(52) U.S. Cl. CPC A61K 35/28; A61P 13/12

(57) ABSTRACT

Mesenchymal stem cells (MSCs) or a pharmaceutical composition comprising a therapeutically effective amount of MSCs can be used in the treatment of chronic kidney disease in felines and canines. Creatinine levels in felines and canines diagnosed with or suffering from chronic kidney disease can be reduced compared to a feline or canine which has not been treated with said MSCs or composition. A pharmaceutical composition comprises MSCs derived from peripheral blood.

### CROSS-REFERENCE TO RELATED APPLICATIONS

This application claims priority to European Patent Application No. 21184504.5 filed Jul. 8, 2021, the disclosure of which is hereby incorporated by reference.

### FIELD OF THE INVENTION

The present invention relates to mesenchymal stem cells for use in the treatment of chronic kidney disease in canines and felines.

### BACKGROUND

Chronic kidney disease (CKD) is the persistent loss of kidney function over time. It is one of the most common conditions affecting older cats and dogs, although it can be seen in animals of any age. Healthy kidneys perform many important functions, most notably filtering the blood and making urine to excrete waste products, maintaining fluid balance in the body, producing certain hormones, and regulating electrolytes. In CKD, all these regulatory processes can be interfered with, and can therefore result in a variety of health problems for an animal.

Animals with CKD may experience a buildup of the waste products and other compounds in the bloodstream that are normally removed or regulated by the kidneys.

This accumulation may make them feel ill and appear lethargic, unkempt, and lose weight. They may also lose the ability to concentrate their urine appropriately, and as a result they may urinate greater volumes and drink more water to compensate. The loss of important proteins and vitamins in their urine may contribute to abnormal metabolism and loss of appetite. They may also experience elevated blood pressure (hypertension), which can affect the function of a number of important systems, including the eyes, brain, and heart. Another cause of lethargy in cats and dogs with CKD is the buildup of acids in their blood. Their affected kidneys may not excrete these compounds appropriately, making the animals prone to blood acidification, or acidosis, a condition that can significantly affect the function of a variety of organ systems in the body. CKD may also decrease an animal's ability to produce red blood cells, which can lead to anemia, a reduced concentration of red blood cells in their blood. This may cause their gums to appear pale pink, or in severe cases, whitish in color, and may make them lethargic.

At the moment, there is no definitive cure for CKD, however some treatments can improve and prolong the lives of these animals suffering from CKD. Therapy is generally geared toward minimizing the buildup of toxic waste products in the bloodstream, maintaining adequate hydration, addressing disturbances in electrolyte concentration, supporting appropriate nutrition, controlling blood pressure, and slowing the progression of kidney disease.

Dietary modification is an important and proven aspect of CKD treatment. However, many cats and dogs have difficulty accepting therapeutic diets, so owners must be patient and dedicated to sticking to the plan. Anemia in a cat or dog with CKD may be treated by replacement therapy with erythropoietin (or with related compounds), which stimulates red blood cell production. In some cases, blood transfusions, which may be used to restore normal red blood cell concentrations using blood obtained from a donor animal, may be necessary.

To date, no known treatments that stop disease progression or repair affected kidneys have been identified. Mesenchymal stem cells (MSC) are being explored as a treatment for CKD in both people and animals, because of their capability to modulate inflammatory responses and mediating cell-cell interactions to promote tissue repair. Several feline and canine studies have investigated their safety and efficacy in treatment and showed interesting results.

The majority of these studies are using autologous MSCs derived from adipose tissue or bone marrow (BM) administered by an intra-articular injection via the renal artery. The production of autologous MSCs for use in treatment, however, requires invasive harvesting of MSCs from each individual patient, followed by time-consuming cultivation of these feline or canine MSCs, which are known to have a relative low culture capacity. In addition, an intra-articular injection is an invasive procedure, which requires sedation, experience and a targeted diagnosis. Therefore, intravenous administration (IV) has been proposed. Rodent models, however, show that the majority of stem cells administered intravenously become trapped in the lungs because the pulmonary capillaries are the first to receive cells after injection. This “first□pass effect,” plus the homing ability of MSCs that draws them to various sites of inflammation in the body, may decrease the total number of MSCs received by the kidneys. Cats treated with IV administered MSCs, although safety of the cell therapy was demonstrated, have not shown improvement in renal function. Studies therefore have been directed away from IV administration again towards intra□arterial delivery of mesenchymal stem cells (MSC), in stromal vascular fraction (SVF) to the kidney in cats with CKD (Thomson, Abigail L et al., 2019).

There thus remains a need in the art for an improved use of MSCs to slow down the disease progression and/or even reverse the pathological condition of chronic kidney disease in the family of cats and dogs. The present invention targets at solving at least one of the aforementioned disadvantages.

### SUMMARY OF THE INVENTION

The present invention and embodiments thereof serve to provide a solution to one or more of above-mentioned disadvantages. To this end, the present invention relates to mesenchymal stem cells (MSCs) or a pharmaceutical composition comprising a therapeutically effective amount of MSCs for use in the treatment of chronic kidney disease (CKD) in felines and canines.

In embodiments, said MSCs are intravenously administered. In embodiments, said MSCs are native MSCs. In embodiments, said MSCs being administered are xenogeneic MSCs. Preferred embodiments of the MSCs for use of the invention are disclosed herein. In a particularly preferred embodiment, in said treatment creatinine levels in felines and canines diagnosed with or suffering from chronic kidney disease are reduced, compared to a feline or canine which has not been treated with said MSCs or composition.

In a second aspect, the present invention relates to a pharmaceutical composition comprising native, peripheral blood-derived MSCs, said MSCs are animal-derived, and present in a sterile liquid.

Intravenous administration is a non-invasive procedure and does not require sedation. Such invasive procedures and/or sedation which may involve risks, especially for older patients which already are at higher risk in developing chronic kidney disease. Therefore, the systemic administration of MSCs via an intravenous (IV) injection offers substantial benefits in therapy application.

### DESCRIPTION OF FIGURES

FIG. 1 shows mean PBMC proliferation before (day 0, T0) and after (week 6, T3) intravenous injection of ten healthy cats with 300.000 ePB-MSCs according to an embodiment of the invention in a mixed lymphocyte reaction (MLR) assay with concanavalin A stimulated feline peripheral blood mononuclear cells (PBMCs).

FIG. 2 shows the measured radioactivity in a cat at different time points following intravenous injection of ^{99m}Tc labelled ePB-MSCs (in DMEM low glucose) according to an embodiment of the invention assessed subjectively through the whole body using a two-headed gamma camera.

### DETAILED DESCRIPTION OF THE INVENTION

The present invention concerns native MSCs for use in the treatment of chronic kidney disease (CKD) in felines and canines, wherein said MSCs may be administered by intravenous injection. An intravenous injection is a non-invasive procedure and does not require sedation. Such invasive procedures and/or sedation which may involve risks, especially for older mammalian patients which already are at higher risk in developing chronic kidney disease. Therefore, the systemic administration of MSCs via an intravenous (IV) injection offers substantial benefits in therapy application.

### Definitions

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any 3, 4, 5, or 7 etc. of said members, and up to all said members.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

The terms “mesenchymal stem cells”, “MSCs” or “mesenchymal stromal cells” refer to multipotent, self-renewing cells that express a specific set of surface antigens and can differentiate into various cell types, including but not limited to adipocytes, chondrocytes, and osteocytes when cultured in vitro or when present in vivo.

The term “isolated”, refers to both the physical identification and isolation of a cells from a cell culture or a biological sample, like blood, that can be performed by applying appropriate cell biology technologies that are either based on the inspection of cell cultures and on the characterization (and physical separation when possible and desired) of cells corresponding to the criteria, or on the automated sorting of cells according to the presence/absence of antigens and/or cell size (such as by FACS). In some embodiments, the terms “isolating” or “isolation” may comprise a further step of physical separation and/or quantification of the cells, especially by carrying out flow cytometry.

The term “in vitro” as used herein denotes outside, or external to, a body. The term “in vitro” as used herein should be understood to include “ex vivo”. The term “ex vivo” typically refers to tissues or cells removed from a body and maintained or propagated outside the body, e.g., in a culture vessel or a bioreactor.

The term “passage” or “passaging” is common in the art and refers to detaching and dissociating the cultured (mesenchymal stem) cells from the culture substrate and from each other. For sake of simplicity, the passage performed after the first time of growing the cells under adherent culture conditions is generally referred to as “first passage” (or passage 1, P1). The cells may be passaged at least one time and preferably two or more times. Each passage subsequent to passage 1 is referred to with a number increasing by 1, e.g., passage 2, 3, 4, 5, or P1, P2, P3, P4, P5, etc.

The term “cell medium” or “cell culture medium” or “medium” refers to an aqueous liquid or gelatinous substance comprising nutrients which can be used for maintenance or growth of cells. Cell culture media can contain serum or be serum-free. The cell medium may comprise or be supplemented with growth factors.

The term “growth factor” as used herein refers to a biologically active substance which influences proliferation, growth, differentiation, survival and/or migration of various cell types, and may effect developmental, morphological and functional changes in an organism, either alone or when modulated by other substances. A growth factor may typically act by binding, as a ligand, to a receptor (e.g., surface or intracellular receptor) present in cells.

“Autologous” administration of MSCs in the present context refers to MSCs from a donor being administered to a recipient, wherein both recipient and donor are the same.

“Allogeneic” administration of MSCs in the present context refers to MSCs from a donor being administered to a recipient, wherein both recipient and donor are of the same species, but are not the same.

“Xenogeneic” administration of MSCs in the present context refers to MSCs from a donor being administered to a recipient, wherein the recipient and the donor are from different species.

“Native MSCs” in the context of the present invention refers to MSCs which have not been exposed to a stimuli environment, such as inflammatory mediators. As used herein, the “inflammatory environment” or “inflammatory condition” refers to a state or condition characterized by (i) an increase of at least one pro-inflammatory immune cell, pro-inflammatory cytokine, or pro-inflammatory chemokine; and (ii) a decrease of at least one anti-inflammatory immune cell, anti-inflammatory cytokine, or anti-inflammatory chemokine.

The term “anti-inflammatory”, “anti-inflammation”, “immunosuppressive”, and “immunosuppressant” refers to any state or condition characterized by a decrease of at least one indication of localized inflammation (such as, but not limited to, heat, pain, swelling, redness, and loss of function) and/or a change in systemic state characterized by (i) a decrease of at least one pro-inflammatory immune cell, pro-inflammatory cytokine, or pro-inflammatory chemokine; and (ii) an increase of at least one anti-inflammatory immune cell, anti-inflammatory cytokine, or anti-inflammatory chemokine.

The “population doubling time” or “PDT” of current invention is to be calculated by the formula: PDT=T/(ln(N_{f}/Nᵢ)/ln(2)), whereby T is the cell culture time (in days) to reach 80% confluency, N_{f} is the final number of cells after cell detachment and whereby Nᵢ is the initial number of cells at time point zero.

By the term “anti-coagulant”, it is meant a composition that can inhibit the coagulation of the blood. Examples of anticoagulants used in the present invention include EDTA or heparin.

The term “buffy coat” in this invention, is to be understood as the fraction of non-coagulated blood, preferably obtained by means of a density gradient centrifugation, whereby the fraction is enriched with white blood cells and platelets.

The term “blood-inter-phase” is to be understood as that fraction of the blood, preferably obtained by means of a density gradient, located between the bottom fraction, mainly consisting of erythrocytes and polymorphonuclear cells, and the upper fraction, mainly consisting of plasma. The blood-interphase is the source of blood mononuclear cells (BMCs) comprising monocytes, lymphocytes, and MSCs.

The term “suspension diameter” as used herein, is understood as the mean diameter of the cells, when being in suspension. Methods of measuring diameters are known in the art. Possible methods are flow cytometry, confocal microscopy, image cytometer, or other methods known in the art.

The term “therapeutically effective amount” is the minimum amount or concentration of a compound or composition that is effective to reduce the symptoms or to ameliorate the condition of a disease.

The term “treatment” refers to both therapeutic, prophylactic or preventive measures to reduce or prevent pathological conditions or disorders from developing or progressing.

“Chronic kidney disease” (CKD) is a type of kidney disease in which there is gradual loss of kidney function over a period of months to years. Chronic renal disease (CRD), chronic renal failure (CRF), and chronic renal insufficiency refer to the same condition. For animals such as cats or dogs, typical visual signs of CKD may include lethargy, weight loss, urinating greater volumes and drinking more water to compensate due to the loss of the ability to concentrate their urine appropriately, loss of appetite, elevated blood pressure (hypertension) affecting eyes, brain and/or heart, and/or pale gums due to a reduction of red blood cells (anemia).

Following diagnosis of CDK, staging of CDK may be undertaken to facilitate appropriate treatment and monitoring of the patient. “IRIS (International Renal Interest Society) stages” are determined based initially on fasting blood creatinine concentration assessed on at least two occasions in a hydrated, stable patient. At stage 1, the patient has a normal blood creatinine level, and at the final stage 4, the patient has increasing risk of systemic clinical signs and uremic crises (see Table 1).

The terms “patient”, “subject”, “animal”, or “mammal” are used interchangeably and refer to a mammalian subject to be treated. Preferably, the mammal is a feline or a canine, such as a cat or a dog.

“Feline” or “felines” in the present invention refers to cats of the Felidae family. A member of this family is also called a felid. The living Felidae are divided in two subfamilies: the Pantherinae and Felinae. Pantherinae includes five Panthera and two Neofelis species, while Felinae includes the other 34 species in ten genera, amongst which domestic cats, cheetahs, servals, lynx' and cougars.

“Canine” or “canines” in the present invention refers to dog-like carnivorans of the Canidae family. A member of this family is called a canid. There are three subfamilies found within the canid family, which are the extinct Borophaginae and Hesperocyoninae, and the extant Caninae. The Caninae are known as canines, and include domestic dogs, wolves, foxes, coyotes, jackals and other extant and extinct species.

“Mixed Lymphocyte Reaction (MLR)” assays are traditionally used to investigate if an external agent stimulates or suppresses T-cell proliferation. By using a MLR assay, the immunomodulatory properties of the MSCs can be investigated. For this MLR assay the responder T-cells are marked with a fluorescent dye which lights up green when it is exposed to a specific light frequency. These responder T-cells are then stimulated with a plant mitogen Concanavalin A (ConA) to induce or stimulate proliferation. ConA is an antigen-independent mitogen and can be used as an alternative T cell stimulus. This lectin is frequently used as a surrogate for antigen-presenting cells in T cell stimulation experiments. Concanavalin A irreversibly binds to glycoproteins on the cell surface and commits T cells to proliferation. This is a quick way to stimulate transcription factors and cytokine production. When the T-cells start to divide the dye is distributed over their daughter cells, so the dye is serially diluting with every cell division. Therefore, the amount of proliferation of T-cells can be measured by looking at the decrease of colour. Thus, to investigate the immunomodulatory properties of the MSCs, these MSCs are added to the stimulated responder T-cells and co-incubated for several days. Appropriate positive and negative controls are included to see if the test is performed successfully. At the end of the incubation period, the amount of T-cell proliferation is measured using flow cytometry, enabling to see whether or not the MSCs suppressed the T-cell proliferation.

### DESCRIPTION

In a first aspect, the present invention relates to mesenchymal stem cells (MSCs) or a pharmaceutical composition comprising a therapeutically effective amount of MSCs for use in the treatment of chronic kidney disease (CKD) in felines and canines, or as a method for treating CKD in felines and canines or for use in the preparation of a medicament for the treatment of CKD in felines and canines, wherein said MSCs are preferably native and preferably intravenously administered.

MSCs have been proposed for use in the treatment of inflammatory-related diseases because of their immunomodulatory properties. These immunomodulatory properties could suppress the exaggerated inflammation process of, amongst others, chronic kidney disease, and slow down its progression on a short term. Previous (feline) studies have investigated their safety and efficacy in the treatment of chronic kidney disease and showed very interesting results. The majority of these animal studies are using autologous MSCs derived from adipose tissue or bone marrow (BM) administered by an intra-articular injection via the renal artery. The production of autologous MSCs for use in treatment, however, requires invasive harvesting of MSCs from each individual patient, followed by time-consuming cultivation of these feline or canine MSCs, which are known to have a relative low culture capacity. In addition, an intra-articular injection is an invasive procedure, which requires sedation, experience and a targeted diagnosis.

Therefore, systemic administration of MSCs may be advantageously achieved via an intravenous (IV) administration, e.g. through injection or infusion, which offers substantial benefits in therapy application. As explained above, rodent models, however, showed that the majority of stem cells administered intravenously become trapped in the lungs because the pulmonary capillaries are the first to receive cells after injection. This “first□pass effect,” plus the homing ability of MSCs that draws them to various sites of inflammation in the body, may decrease the total number of MSCs received by the kidneys. Cats treated with IV administered MSCs in previous studies did not show improvement in renal function. Studies therefore have been directed away from IV administration again towards invasive intra□arterial delivery of mesenchymal stem cells (MSC), in stromal vascular fraction (SVF) to the kidney in cats with CKD (Thomson, Abigail L et al., 2019).

Said feline may be any cat of the Felidae family, preferably of the Felinae subfamily, more preferably a domestic cat (Fells catus). Said canine may be any dog-like carnivoran of the Canidae family, preferably of the Caninae subfamily, more preferably a domestic dog (Canis familiaris).

In an embodiment, said MSCs for use are native. Such native MSCs have not first in vitro been exposed to a stimulating agent, such as inflammatory mediators or an inflammatory environment. Such inflammatory environment refers to a state or condition characterized by (i) an increase of at least one pro-inflammatory immune cell, pro-inflammatory cytokine, or pro-inflammatory chemokine; and (ii) a decrease of at least one anti-inflammatory immune cell, anti-inflammatory cytokine, or anti-inflammatory chemokine. The use of native MSCs is a favorable option as they allow the production of a ready-to-use product, with minimum manufacturing and handling, thereby lowering cost of production.

By preference, the MSCs have a cell size between 10 μm to 100 μm, more preferably between 15 μm and 80 μm, more preferably between 20 μm and 75 μm, more preferably between 25 μm and 50 μm. In an embodiment, the MSCs for use according to the current invention are selected by size by means of a filter system, wherein the cells are run through a double filtration step using a 40 μm filter. Double or multiple filtration steps are preferred. The latter provides for a high population of single cells and avoids the presence of cell aggregates. Such cell aggregates may cause cell death during the preservation of the cells by freezing and may all have an impact on further downstream applications of the cells. For instance, cell aggregates may higher the risk of the occurrence of a capillary embolism when administered intravenously.

The MSCs for use according to the present invention may originate from various tissues or body fluids, in particular from blood, bone marrow, fat tissue or amniotic tissue. Bone marrow harvesting of MSCs has been reportedly associated with haemorrhage, chronic pain, neurovascular injury, and even death. Adipose tissue as a source for MSCs is regarded as a safer option. However, harvesting of MSCs from adipose tissue still requires an incision in the donor animal, hence this is still an invasive procedure. MSCs derived from blood show similar morphology as MSCs derived from bone marrow and adipose tissue. As a consequence, by preference, the MSCs originate from blood, including but not limited to umbilical cord blood and peripheral blood. More preferably, the MSCs originate from peripheral blood. Blood is not only a non-invasive and painless source, but also simple and safe to collect and, consequently, easily accessible. The MSCs or blood comprising MSCs may originate from all mammals, including, but not limited to, humans, domestic and farm animals, zoo animals, sport animals, pet animals, companion animals and experimental animals, such as, for example, mice, rats, rabbits, dogs, cats, cows, horses, pigs and primates, e.g., monkeys and apes;

especially horse, human, cat, dogs, rodents, etc. In an embodiment, said origin is equine. In particular MSCs may be derived from peripheral blood, preferably equine peripheral blood, which allows multiple MSC collections per year with minimal discomfort or morbidities for the donor animal.

In some cases, the use of allogeneic or xenogeneic MSCs is a more favorable option as they offer a stringent selection of healthy and high-quality stem cell donors. They allow the production of a ready-to-use product, avoiding the invasive harvesting and time-consuming cultivation of MSCs from each individual patient. Because of the relative low culture capacity of feline and canine MSCs compared to for example equine or human MSCs, the use of xenogeneic (e.g. human or equine) MSCs is preferred above allogeneic feline or canine MSCs, especially for commercial applications, such as for use in the treatment of CKD in felines and canines.

Therefore, in a particular embodiment the MSCs of the current invention may be used for allogeneic or xenogeneic administration to a subject. As already indicated, allogeneic or xenogeneic use allows a better control of the quality of the MSCs, as different donors may be screened, and the optimal donors may be selected. The latter is indispensable in view of preparing functional MSCs. This is in contrast to autologous use of MSCs, as in this case, quality of the cells is more difficult to be ensured. Nonetheless, autologous use may have his benefits as well. In one case, blood MSCs are isolated, for which blood from a donor was used who was later also recipient of the isolated MSCs. In another case, blood is used from donors in which the donor is preferably of the same family, gender or race as the recipient of the MSCs isolated from the blood of donors. In particular, these donors will be tested on common current transmittable diseases or pathologies, in order to avoid the risk of horizontal transmission of these pathologies or diseases through the stem cells. Preferably, the donors/donor animals are kept in quarantine. When using donor horses they can be, for example tested for the following pathologies, viruses or parasites: equine infectious anemia (EIA), equine rhinopneumonitis (EHV-1, EHV-4), equine viral arteritis (EVA), West Nile virus (WNV), African horse Sickness (AHS), dourine (Trypanosoma), equine piroplasmosis, glanders (malleus, glanders), equine influenza, Lyme borreliosis (LB) (Borrelia burgdorferi, Lyme disease).

In an embodiment, the MSCs for use of the present invention may be characterized by the presence of/are measured positive for one or more of the following markers CD29, CD44, CD90, CD105, vimentin, fibronectin, Ki67, CK18 or any combination thereof. In a further embodiment, the MSCs for use of current invention may be characterized by the presence of mesenchymal markers CD29, CD44 and CD90. By means of the latter, the purity of the obtained MSCs can be analyzed, and the percentage of MSCs can be determined.

CD29 is a cell surface receptor encoded by the integrin beta 1 gene, wherein the receptor forms complexes with other proteins to regulating physiological activities upon binding of ligands. The CD44 antigen is a cell surface glycoprotein involved in cell-cell interactions, cell adhesion and migration. In addition, is CD44 a receptor for hyaluronic acid and can also interact with other ligands such as osteopontin, collagens and matrix metalloproteinases (MMPs). The CD90 antigen is a conserved cell surface protein considered as a marker for stem cells, like MSCs. The MSCs of current invention being triple positive for CD29/CD44/CD90 enables the person skilled in the art for a fast and unambiguous selection of the MSCs and provides the MSCs biological properties which are of interest for further downstream applications.

In an embodiment, the MSCs for use of the current invention are characterized by the absence of/measure negative for Major Histocompatibility Complex (MHC) class II molecules, preferably all currently known MHC Class II molecules, classifying the cell as a cell that can be used in cellular therapy for mammalians, such as feline and canine cellular therapy. Even when the MSCs are partly differentiated, the MSCs remain negative for MHC class II molecules. Detecting presence or absence, and quantifying the expression of MHC II molecules can be performed using flow cytometry.

In another and further embodiment the MSCs measure negative for CD45 antigen, a marker for hematopoietic cells.

In an embodiment, the MSCs measure negative for both MHC class II molecules and CD45.

In a particularly preferred embodiment, the MSCs for use of the current invention measure positive for mesenchymal markers CD29, CD44 and CD90 and measure negative for MHC class II molecules and CD45.

MSCs in general express MHC Class I antigen on their surface. In a particular embodiment the MSCs for use of current invention have a low or undetectable level of the MHC Class I marker. In a most preferred embodiment said MSCs measure negative for MHC Class II markers and have a low or undetectable level of MHC Class I marker, wherein said cell exhibits an extremely low immunogenic phenotype. For the sake of the current invention, said low level should be understood as less than 25%, more preferably less than 15% of the total cells expressing said MHC I or MHC II. Detecting presence or absence, and quantifying the expression of MHC I and MHC II molecules can be performed using flow cytometry.

These immunological properties of the MSCs limit the ability of the recipient immune system to recognize and reject cells, preferably allogeneic or xenogeneic cells, following cellular transplantation. The production of factors by MSCs, that modulate the immune response together with their ability to differentiate into appropriate cell types under local stimuli make them desirable stem cells for cellular therapy.

In an embodiment, the MSCs for use of the invention, secrete immunomodulatory prostaglandin E2 cytokine when present in an inflammatory environment or condition.

Inflammatory environments or conditions are characterized by the recruitment of immune cells of the blood. Inflammatory mediators include prostaglandins, inflammatory cytokines such as IL-1β, TNF-α, IL-6 and IL-15, chemokines such as IL-8 and other inflammatory proteins like TNF-α, IFN-γ. These mediators are primarily produced by monocytes, macrophages, T-cells, B-cells to recruit leukocytes at the site of inflammation and subsequently stimulate a complex network of stimulatory and inhibitory interactions to simultaneously destruct and heal the tissue from the inflammatory process.

Prostaglandin E2 (PgE2) is a subtype of the prostaglandin family. PgE2 is synthesized from arachidonic acid (AA) released from membrane phospholipids through sequential enzymatic reactions. Cyclooxygenase-2 (COX-2), known as prostaglandin-endoperoxidase synthase, converts AA to prostaglandin H2 (PgH2), and PgE2 synthase isomerizes PgH2 to PgE2. As a rate-limiting enzyme, COX-2 controls PgE2 synthesis in response to physiological conditions, including stimulation by growth factors, inflammatory cytokines and tumor promoters.

In a particular embodiment, said MSCs present in an inflammatory environment secrete the soluble immune factor prostaglandin E2 (PgE2) in a concentration ranging between 10³ to 10⁶ picogram per ml to induce or stimulate MSC-regulated immunosuppression.

The PgE2 secretion of the MSCs in those specific concentration ranges stimulates anti-inflammatory processes in vitro and together with their ability to differentiate into appropriate cell types makes them desirable for cellular transplantation.

In a preferred embodiment the MSCs for use of the current invention measures:
positive for mesenchymal markers CD29, CD44 and CD90;
positive for one or more markers comprised in the group consisting of vimentin, fibronectin, Ki67, or a combination thereof;
negative for MHC class II molecules;
negative for hematopoietic marker CD45, and
preferably have a low or undetectable level of MHC Class I molecules, wherein said low level should be understood as less than 25%, more preferably less than 15% of the total cells expressing MHC I.

In a most preferred embodiment, the MSCs for use of the current invention measures:
positive for mesenchymal markers CD29, CD44 and CD90;
positive for one or more markers comprised in the group consisting of vimentin, fibronectin, Ki67, or a combination thereof;
negative for MHC class II molecules;
negative for hematopoietic marker CD45; and
preferably have a low or undetectable level of MHC Class I molecules, wherein said low level should be understood as less than 25%, more preferably less than 15% of the total cells expressing MHC I,
   wherein said cell secretes immunomodulatory PgE2 cytokine in a concentration ranging between 10³ to 10⁶ picogram per ml when present in an inflammatory environment or condition.

In another or further embodiment, the MSCs for use according to the invention, have an increased secretion of at least one of the molecules chosen from IL-6, IL-10, TGF-beta, NO or a combination thereof, and a decreased secretion of IL-1 when present in an inflammatory environment or condition, and compared to an MSC having the same characteristics but not being subjected to said inflammatory environment or condition.

In a preferred embodiment, the MSCs have an increased secretion of at least one of the molecules chosen of IL-6, IL-10, TGF-β, NO, or a combination thereof, and a decreased secretion of IL-1 when present in an inflammatory environment or condition. Comparison can be made with a mesenchymal stem cell having the same characteristics as presented above, but which is not subjected to said inflammatory environment or condition.

Preferably the MSCs have an increased secretion of PgE2 in combination with two or more of the abovementioned factors.

PgE2, IL-6, IL-10, TGF-β and NO help suppressing the proliferation and function of major immune cell populations like T cells and B cells. In addition, the MSCs express low levels of MHC class I molecules and/or are negative for MHC class II molecules on their surface, escaping immunogenic reactions. In addition, the MSCs of current can suppress the proliferation of white blood cells by their increased secretion of abovementioned factors, once again helping to avoid immunogenic reactions of the host.

In another or further embodiment the MSCs stimulate the secretion of PgE2, IL-6, IL-10, NO, or a combination thereof and/or suppress the secretion of TNF-α, IFN-γ, IL-1, IL-13, or a combination thereof in the presence of peripheral blood mononuclear cells (PBMCs). In another or further embodiment, the MSCs suppress the secretion of TGF-β1 in the presence of PBMCs.

In the inflammatory environment the MSCs secrete multiple factors that modulate the immune response of the host. In addition, the MSCs have the stimulatory effect to induce or stimulate the secretion of one or more factors selected from the group consisting of PgE2, IL-6, IL-10, NO, or a combination thereof. Next to the stimulatory effect of the MSCs on the PBMCs in an inflammatory environment, the MSCs also have an suppressive effect on the secretion of the PBMCs, resulting in a decrease of one or more factors selected from the group consisting of TNF-α, IFN-γ, IL-1, TGF-β1, IL-13, or a combination thereof. The MSCs have a regulatory effect in the inflammatory environment, making them useful in the treatment of all sorts of diseases, particularly disorders of the immune system.

In general, any technology for identifying and characterizing cellular markers for a specific cell type (e.g. mesenchymal, hepatic, hematopoietic, epithelial, endothelial markers) or having a specific localization (e.g. intracellular, on cell surface, or secreted) that are published in the literature may be considered appropriate for characterizing MSCs. Such technologies may be grouped in two categories: those that allow maintaining cell integrity during the analysis, and those based on extracts (comprising proteins, nucleic acids, membranes, etc.) that are generated using such cells. Among the technologies for identifying such markers and measuring them as being positive or negative, immunocytochemistry or analysis of cell culture media are preferred since these allow marker detection even with the low amount of cells, without destroying them (as it would be in the case of Western Blot or Flow Cytometry).

Immunomodulatory properties of MSCs may be assayed using an MLR assay. For this MLR assay responder T-cells are marked with a fluorescent dye which lights up green when it is exposed to a specific light frequency. These responder T-cells are then stimulated with a plant mitogen (ConA) to induce or stimulate proliferation. When the T-cells start to divide the dye is distributed over their daughter cells, so the dye is serially diluting with every cell division. Therefore, the amount of proliferation of T-cells can be measured by looking at the decrease of color. Thus, to investigate the immunomodulatory properties of the MSCs, these MSCs are added to the stimulated responder T-cells and co-incubated for several days. Appropriate positive and negative controls are included to see if the test is performed successfully. At the end of the incubation period, the amount of T-cell proliferation is measured using flow cytometry, enabling us to see whether or not the MSCs suppressed the T-cell proliferation.

Relevant biological features of the MSCs can be identified by using technologies such as flow cytometry, immunocytochemistry, mass spectrometry, gel electrophoresis, an immunoassay (e.g. immunoblot, Western blot, immunoprecipitation, ELISA), nucleic acid amplification (e.g. real time RT-PCR), enzymatic activity, omics-technologies (proteomics, lipidomics, glycomics, translatomics, transcriptomics, metabolomics) and/or other biological activity.

The MSCs of current invention may be derived by any standard protocol known in the art. In an embodiment, said MSCs may be obtained via a method wherein the MSCs are isolated from blood or a blood phase and wherein said cells are cultured and expanded in a basal medium, preferably a low glucose medium.

Basal medium formulation as known in the art include, but are not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, Medium 199, Waymouth's 10 MB 752/1 or Williams Medium E, and modifications and/or combinations thereof. Compositions of the above basal media are generally known in the art and it is within the skill of one in the art to modify or modulate concentrations of media and/or media supplements as necessary for the cells cultured. A preferred basal medium formulation may be one of those available commercially such as DMEM, which are reported to sustain in vitro culture of MSCs, and including a mixture of growth factors for their appropriate growth, proliferation, maintenance of desired markers and/or biological activity, or long-term storage.

Such basal media formulations contain ingredients necessary for mammal cell development, which are known per se. By means of illustration and not limitation, these ingredients may include inorganic salts (in particular salts containing Na, K, Mg, Ca, Cl, P and possibly Cu, Fe, Se and Zn), physiological buffers (e.g., HEPES, bicarbonate), nucleotides, nucleosides and/or nucleic acid bases, ribose, deoxyribose, amino acids, vitamins, antioxidants (e.g., glutathione) and sources of carbon (e.g. glucose, pyruvate, e.g., sodium pyruvate, acetate, e.g., sodium acetate), etc. It will also be apparent that many media are available as low-glucose formulations with or without sodium pyruvate.

Method for isolating MSCs from blood or a blood phase and culturing and expanding said cells are known in the art and for instance described in WO2014053418 or WO2014053420.

In an embodiment, such method for isolating MSCs from blood or a blood phase and culturing and expanding said cells in a low glucose medium may comprise the following steps:
a) the collection of one or more blood samples from donors, in a sample vial, coated with an anti-coagulant;
b) centrifuging the blood samples to obtain a 3-phase distribution, consisting of a plasma-phase, buffy coat, and erythrocytes phase;
c) collecting the buffy coat and loading it on a density gradient;
d) collecting of the blood-inter-phase obtained from the density gradient of step c);
e) isolating of MSCs from the blood-inter-phase by centrifugation;
f) seeding between 2.5×10⁵/cm² and 5×10⁵/cm² MSCs in culture and keeping them in a low glucose growth medium supplemented with dexamethasone, antibiotics and serum.

In an embodiment, anticoagulants may be supplemented to the MSCs. Non-limiting examples are EDTA or heparin.

The number of seeding is crucial to ultimately obtain a pure and viable population MSCs at an acceptable concentration, as a too dense seeding will lead to massive cell death during expansion and a non-homogenous population of MSCs and a too dispersed seeding will result in little or no colony formation of MSCs, so that expansion is not or hardly possible, or it will take too much time. In both cases the viability of the cells will be negatively influenced.

In a preferred embodiment of current invention, the MSCs have a high cell viability, wherein at least 90%, more preferably at least 95%, most preferably 100% of said cells are viable.

The blood-interphase is the source of blood mononuclear cells (BMCs) comprising monocytes, lymphocytes, and MSCs. By preference, the lymphocytes are washed away at 37° C., while the monocytes die within 2 weeks in the absence of cytokines necessary to keep them alive. In this way, the MSCs are purified. The isolation of the MSCs from the blood-inter-phase is preferably done by means of centrifugation of the blood-inter-phase, after which the cell pellet is washed at least once with a suitable buffer, such as a phosphate buffer.

In a further embodiment the MSCs of current invention are negative for monocytes and macrophages, both within a range between 0% and 7.5%.

In particular, the mesenchymal cells are kept at least 2 weeks in growth medium. Preferably, growth medium with 1% dexamethasone is used, as the specific characteristics of the MSCs are kept in said medium.

Following a minimum period of 2 weeks (14 days), preferably 3 weeks (21 days) MSC colonies will become visible in the culture bottles. In a subsequent step g) at least 6×10³ stem cells/cm² are transferred to an expansion medium containing low glucose, serum and antibiotics for the purpose of expanding the MSCs. Preferably, the expansion of the MSCs will occur in minimal five cell passages. In this way sufficient cells can be obtained. Preferably, the cells are split at 70% to 80% confluency. The MSCs can be maintained up to 50 passages in culture. After this the risk of loss in vitality, senescence or mutation formation occurs.

In a further embodiment, the population doubling time (PDT) between each passage during expansion of the MSCs should be between 0.7 and 3 days after trypsinization. Said PDT between each passage during expansion of the MSCs is preferably between 0.7 and 2.5 days after trypsinization.

In a preferred embodiment, the MSCs for use according to the invention have a spindle-shaped morphology. The morphological characterization of the MSCs of current invention classifies the cell as an elongated, fibroblast-like, spindle-shaped cell. This type of cell is distinct form other populations of MSCs with small self-renewing cells which reveal mostly a triangular or star-like cell shape and populations of MSCs with a large, cuboidal or flattened pattern with a prominent nucleus. The selection of MSCs with this specific morphological characteristic along with the biological markers enables the person skilled in the art to isolate the MSCs of current invention. A morphological analysis of cells can easily be performed by a person skilled in the art using phase contrast microscopy. Besides, the size and granularity of MSCs can be evaluated using forward and side scatter diagram in flow cytometry or other techniques known by a person skilled in the art.

In another or further preferred embodiment, the MSCs have a suspension diameter between 10 μm and 100 μm. The MSCs for use of current invention have been selected based on size/suspension diameter. By preference, the MSCs have a cell size between 10 to 100 μm, more preferably between 15 and 80 μm, more preferably 20 and 75 μm, more preferably between 25 and 50 μm. Preferably, the selection of cells based on cell size occurs by a filtration step. For instance, MSCs with a cell concentration ranging between 10³ to 10⁷ MSCs per ml, wherein said cells are preferably diluted in low glucose DMEM medium, are selected by size by means of a filter system, wherein the cells are run through a double filtration step using a 40 μm filter. Double or multiple filtration steps are preferred. The latter provides for a high population of single cells and avoids the presence of cell aggregates. Such cell aggregates may cause cell death during the preservation of the cells by freezing and may all have an impact on further downstream applications of the cells. For instance, cell aggregates may higher the risk of the occurrence of a capillary embolism when administered intravenously.

In an embodiment, said therapeutically effective amount of MSCs is between 10⁵-10⁷ MSCs in said composition.

In a preferred embodiment, the MSCs for use according to the present invention are formulated for administration in a subject by means of intravenous injection or infusion.

In an embodiment, a therapeutically effective amount of MSCs is administered to the feline or canine patient, preferably a dose of 10⁵-10⁷ MSCs per patient is administered. In an embodiment, a single dose is administered.

The minimum therapeutically effective dose that yields a therapeutic benefit to a subject is at least 10⁵ of the MSCs per administration. Preferably, each administration is by intravenous injection and comprises between 10⁵ to 5×10⁵ MSCs per administration, wherein said MSCs preferably are native and/or xenogeneic.

In an embodiment, said MSCs are administered at least twice, at least three times, at least four times, at least five times, preferably with intervals.

In another or further embodiment, the treatment further comprises: multiple administrations of the MSCs or the composition comprising MSCs, for example multiple intravenous administrations, doses of 10⁵-10⁷ MSCs per feline or canine patient, wherein said multiple doses are administered at various time points, including but not limited to one or more of the following time points 1 day apart, 2 days apart, 3 days apart, 4 days apart, 5 days apart, 6 days apart, 7 days (1 week) apart, 2 weeks apart, 3 weeks apart, 4 weeks apart, 5 weeks apart, 6 weeks apart, 7 weeks apart, 8 weeks apart, 3 months apart, 6 months, 9 months apart, and/or 1 year apart. Preferably each dose is administered at least 2 weeks apart, more preferably at least 3 weeks apart, even more preferably at least 4 weeks apart, and most preferably at least 6 weeks apart.

In an embodiment, said composition comprises said MSCs present in a sterile liquid. A non-limiting example of such sterile liquid is a minimal essential medium (MEM), such as Dulbecco's Modified Eagle Medium (DMEM). Said sterile liquid should be safe for intravenous administration, e.g. via injection or infusion, to a mammalian patient.

As non-limiting examples, said sterile liquid is a minimal essential medium, such as a basal medium. Basal medium formulation as known in the art include, but are not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, Medium 199, Waymouth's 10 MB 752/1 or Williams Medium E, and modifications and/or combinations thereof. Compositions of the above basal media are generally known in the art and it is within the skill of one in the art to modify or modulate concentrations of media and/or media supplements as necessary for the cells cultured. A preferred basal medium formulation may be one of those available commercially such as DMEM, which are reported to sustain in vitro culture of MSCs, and including a mixture of growth factors for their appropriate growth, proliferation, maintenance of desired markers and/or biological activity, or long-term storage.

Such basal media formulations contain ingredients necessary for mammal cell development, which are known per se. By means of illustration and not limitation, these ingredients may include inorganic salts (in particular salts containing Na, K, Mg, Ca, Cl, P and possibly Cu, Fe, Se and Zn), physiological buffers (e.g., HEPES, bicarbonate), nucleotides, nucleosides and/or nucleic acid bases, ribose, deoxyribose, amino acids, vitamins, antioxidants (e.g., glutathione) and sources of carbon (e.g. glucose, pyruvate, e.g., sodium pyruvate, acetate, e.g., sodium acetate), etc. It will also be apparent that many media are available as low-glucose formulations with or without sodium pyruvate.

By preference, said composition comprises at least 75%, more preferably at least 80%, even more preferably at least 85%, most preferably at least 90% of single cells and whereby said single cells have a suspension diameter of between 10 μm and 100 μm, more preferably between 15 μm and 80 μm, more preferably between 20 μm and 75 μm, more preferably between 25 μm and 50 μm. As previously mentioned, the diameter of the cells as well as their single-cell nature is crucial for any downstream application, e.g. intravenous administration, and for the vitality of the cells.

By preference, said composition comprises at least 90% MSCs, more preferably it will comprise at least 95% MSCs, more preferably at least 99%, most preferably 100% MSCs.

The volume and concentration of the composition in the form of a sterile liquid comprising the MSCs is preferably adapted for intravenous injection. In an embodiment, the pharmaceutical composition may be administered to the animal in the form of a sterile liquid comprising, after final adjustment, the MSCs at a concentration of 10⁵-10⁷ cells per mL.

In an embodiment, with each intravenous injection or infusion, a therapeutically effective amount of MSCs is administered, preferably each injection or infusion comprises a dose of 10⁵ to 10⁷ of said MSCs.

In an embodiment, the pharmaceutical composition comprises a therapeutically effective of amount of MSCs of between 10⁵-10⁷ MSCs per mL, preferably 10⁵ to 10⁶ MSCs per mL, more preferably 10⁵-5×10⁵ MSCs per mL of said composition, most preferably 3×10⁵ MSCs per mL of said composition.

In an embodiment, one dosage of said composition has a volume of about 0.5 to 5 ml, preferably of about 0.5 to 5 ml, preferably of about 0.5 to 3 ml, preferably of about 0.5 to 2 ml, more preferably of about 0.5 to 1.5 ml, most preferably of about 1 ml. In another or further embodiment, one dosage of said composition has a volume of maximally about 5 ml, preferably maximally about 4 ml, more preferably maximally about 3 ml, more preferably maximally about 2 ml, most preferably said volume is about 1 ml. This amount is suitable for intravenous administration.

Said dosage may be formulated in a vial or in a pre-filled syringe.

In an embodiment, the volume of the composition which is administered per injection to a patient is adapted in accordance with the patient's body weight. In another an embodiment, a fixed dose of 10⁵-10⁷ MSCs per patient, preferably 10⁵ to 10⁶ MSCs, more preferably 10⁵-5×10⁵ MSCs, most preferably 3×10⁵ MSCs is administered.

The inventors have further discovered that a particularly effective treatment is achieved by a dosing regimen comprising at least two dosages of the MSCs for use or the pharmaceutical composition for use as described above in any of the embodiments.

Therefore, a further embodiment relates to a pharmaceutical composition for use in the treatment of CKD in felines and canines, wherein:
the treatment comprises a step of administering, preferably intravenously, a first amount of said composition comprising a total dose of 10⁵-10⁷ MSCs per patient, and
the treatment further comprises a step of administering, preferably intravenously, a second amount of said composition, said second amount comprising a second total dose of 10⁵-10⁷ MSCs, wherein said MSCs preferably are native and/or xenogeneic, and
   wherein said second dose is administered 1 day after the first amount, 2 days after the first amount, 3 days after the first amount, 4 days after the first amount, 5 days after the first amount, 6 days after the first amount, 7 days (1 week) after the first amount, 2 weeks after the first amount, 3 weeks after the first amount, 4 weeks after the first amount, 5 weeks after the first amount, 6 weeks after the first amount, 7 weeks after the first amount, 8 weeks after the first amount, 3 months after the first amount, 6 months, 9 months after the first amount, and/or 1 year after the first amount. Preferably each dose is administered at least 2 weeks after the first amount, more preferably at least 3 weeks after the first amount, even more preferably at least 4 weeks after the first amount, and most preferably at least 6 weeks after the first amount.

In an embodiment, said second dose is identical to the first dose. In another embodiment, said second dose is lower than the first dose. In yet another embodiment, said second dose is higher than the first dose.

In an embodiment, a third, fourth and/or even a fifth amount of said composition may be administered, preferably intravenously, to said patient, wherein said third, fourth and/or fifth amount comprises a third, fourth and/or fifth total dose of 10⁵-10⁷ MSCs, wherein said MSCs preferably are native and/or xenogeneic.

In an embodiment, a sixth or more amount of said composition may be administered, preferably intravenously, to said patient, wherein said sixth or more amount comprises a sixth or more total dose of 10⁵-10⁷ MSCs, wherein said MSCs preferably are native and/or xenogeneic.

Upon diagnosis of CDK in felines and canines, staging of CDK may be undertaken to facilitate appropriate treatment and monitoring of the patient. IRIS stages are determined based initially on fasting blood creatinine concentration assessed on at least two occasions in a hydrated, stable patient. At stage 1, the patient has a normal blood creatinine level (<1.6 mg creatinine/dl for cats, <1.4 mg/creatinine/dl for dogs), and at the final stage 4 (>5.0 mg creatinine/dl), the patient has increasing risk of systemic clinical signs and uremic crises.

The present invention therefore also relates to the MSCs or the pharmaceutical composition comprising an effective amount of MSCs for use as described in any of the previous embodiments, wherein creatinine levels in felines and canines diagnosed with or suffering from chronic kidney disease are reduced, compared to a feline or canine which has not been treated with said MSCs or composition. Said MSCs or composition are preferably intravenously administered, and said MSCs are preferably native and/or xenogeneic.

In an embodiment, the mean creatinine levels of said felines or canines receiving the MSCs or the pharmaceutical composition are reduced with at least 1%, preferably at least 2%, preferably at least 3%, preferably at least 4%, preferably at least 5%, preferably at least 6%, preferably at least 7%, preferably at least 8%, preferably at least 9%, preferably at least 10%, preferably at least 11%, preferably at least 12%, preferably at least 13%, preferably at least 14%, preferably at least 15%, preferably at least 16%, preferably at least 17%, preferably at least 18%, preferably at least 19%, preferably at least 20%, preferably at least 21%, preferably at least 22%, preferably at least 23%, preferably at least 24%, preferably at least 25%, preferably at least 30%, compared to the mean creatinine levels of felines or canines which have not been treated with said MSCs or composition.

By administering the MSCs or the pharmaceutical composition comprising a therapeutically effective amount of MSCs, to felines and canines diagnosed with or suffering from CDK, one or more symptoms of CDK, such as increased creatinine levels, can be reduced, mitigated, ameliorated and/or reversed in said patients suffering from CDK compared to said symptoms prior to administration of said MSCs or said pharmaceutical composition comprising MSCs to said patients.

In an embodiment, the mean creatinine levels of said felines or canines are reduced with at least 1%, preferably at least 2%, preferably at least 3%, preferably at least 4%, preferably at least 5%, preferably at least 6%, preferably at least 7%, preferably at least 8%, preferably at least 9%, preferably at least 10%, preferably at least 11%, preferably at least 12%, preferably at least 13%, preferably at least 14%, preferably at least 15%, preferably at least 16%, preferably at least 17%, preferably at least 18%, preferably at least 19%, preferably at least 20%, preferably at least 21%, preferably at least 22%, preferably at least 23%, preferably at least 24%, preferably at least 25%, preferably at least 30%, compared to the mean creatinine levels of said felines or canines prior to administration of said MSCs or said pharmaceutical composition comprising MSCs.

In an embodiment, by administering the MSCs or the pharmaceutical composition comprising a therapeutically effective amount of MSCs, to a feline or canine diagnosed with or suffering from CDK, the quality of life (QoL) of said feline or canine is improved compared to the quality of life of said feline or canine prior to administration of said MSCs or said pharmaceutical composition comprising MSCs.

Said quality of life can be for instance determined by means of a linear analog scale, asking owners to rate their pets' QoL on a scale of 1-10. When 1 being the best quality of life and 10 being the worst quality of life, improving the quality of life thus refers to obtaining a lower score on said scale. When 1 being the worst quality of life and 10 being the best quality of life, improving the quality of life thus refers to obtaining a higher score on said scale.

In an embodiment, the quality of life (QoL) of said feline or canine is measured by a linear analog scale and the value thus obtained is improved with at least 10%, preferably at least 15%, preferably at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, such as 86%, compared to the value of said feline or canine prior to administration of said MSCs or said pharmaceutical composition comprising MSCs. An “improved value” refers to a better quality of life.

Said quality of life can also be calculated by a scoring scheme or a questionnaire.

In an embodiment, the quality of life of felines is calculated by means of the quality of life (QoL) tool discussed in the article of Bijsmans et al. (Bijsmans E S, Jepson R E, Syme H M, Elliott J, Niessen S J. Psychometric Validation of a General Health Quality of Life Tool for Cats Used to Compare Healthy Cats and Cats with Chronic Kidney Disease. J Vet Intern Med. 2016 January-February; 30(1): 183-91. doi: 10.1111/jvim.13656. Epub 2015 Nov. 14. PMID: 26567089; PMCID: PMC4913638.). In brief, the questionnaire is divided into 4 domains: general health (GH), eating (E), behavior (B) and management (M). Each item is scored according to the frequency or severity with which it impacted the cat's life, and an importance rating is included for all questions to capture individual differences. The frequency or severity ratings range from −3 to +3, and the importance ratings range from 0 to +3. At the end of the calculation series, this results in an average-weighted score that provides an overall quantitative measure of the cat's Quality of Life.

In an embodiment, the quality of life of canines is calculated by means of the quality of life (QoL) questionnaire discussed in the article of Lavan (Lavan R P. Development and validation of a survey for quality of life assessment by owners of healthy dogs. Vet J. 2013 September; 197(3): 578-82. doi: 10.1016/j.tvjl.2013.03.021. Epub 2013 Apr. 29. PMID: 23639368.). In an embodiment, the quality of life of canines is calculated by means of an adapted version of the quality of life (QoL) questionnaire discussed in the aforementioned article of Lavan.

In an embodiment, the quality of life (QoL) of said feline or canine is measured by a scoring scheme and the score thus obtained is improved with at least 10%, preferably at least 15%, preferably at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, such as 86%, compared to the quality of life score of said feline or canine prior to administration of said MSCs or said pharmaceutical composition comprising MSCs. An “improved score” refers to a better quality of life.

In an embodiment, the quality of life (QoL) of said feline diagnosed with or suffering from CDK is calculated by means of the quality of life (QoL) tool discussed in the article of Bijsmans et al. and the score thus obtained is improved with at least 10%, preferably at least 15%, preferably at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, such as 86%, compared to the quality of life score of said feline prior to administration of said MSCs or said pharmaceutical composition comprising MSCs. An “improved score” refers to a better quality of life.

In an embodiment, by administering the MSCs or the pharmaceutical composition comprising a therapeutically effective amount of MSCs, to felines or canines diagnosed with or suffering from CDK, the quality of life (QoL) of said felines or canines is improved compared to the quality of life of felines or canines which have not been treated with said MSCs or composition.

In an embodiment, the quality of life (QoL) of said felines or canines is measured by a linear analog scale and the mean value thus obtained is improved with at least 10%, preferably at least 15%, preferably at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, such as 86%, compared to the mean value of said felines or canines which have not been treated with said MSCs or composition.

In an embodiment, the quality of life (QoL) of said felines or canines is measured by a scoring scheme and the mean score thus obtained is improved with at least 10%, preferably at least 15%, preferably at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, such as 86%, compared to the mean quality of life score of said felines or canines which have not been treated with said MSCs or composition.

In an embodiment, the quality of life (QoL) of said felines diagnosed with or suffering from CDK is measured by means of the quality of life (QoL) tool discussed in the article of Bijsmans et al. and the mean score thus obtained is improved with at least 10%, preferably at least 15%, preferably at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, such as 86%, compared to the mean quality of life score of said felines which have not been treated with said MSCs or composition.

In a last aspect, the present invention relates to a specific pharmaceutical composition comprising peripheral blood-derived MSCs. Said composition comprises native peripheral blood-derived MSCs, said MSCs are animal-derived, preferably mammal-derived, and present in a sterile liquid at a concentration of between 10⁵-10⁷ MSCs per mL of said composition, wherein one dosage of said composition has a volume of about 0.5 to 5 ml, wherein said MSCs measure positive for mesenchymal markers CD29, CD44 and CD90 and measure negative for MHC class II molecules and CD45, and wherein said MSCs have a suspension diameter between 10 μm and 100 μm.

In an embodiment, said pharmaceutical composition is intravenously administered. In a preferred embodiment, said MSCs are equine derived.

In an embodiment, said one dosage of said composition has a volume of about 0.5 to 5 ml, preferably of about 0.5 to 5 ml, preferably of about 0.5 to 3 ml, preferably of about 0.5 to 2 ml, more preferably of about 0.5 to 1.5 ml, most preferably of about 1 ml. In another or further embodiment, one dosage of said composition has a volume of maximally about 5 ml, preferably maximally about 4 ml, more preferably maximally about 3 ml, more preferably maximally about 2 ml, most preferably said volume is about 1 ml. This amount is suitable for intravenous administration.

In another or further preferred embodiment, the MSCs have a suspension diameter between 15 and 80 μm, more preferably 20 and 75 μm, more preferably between 25 and 50 μm.

A person of ordinary skill will appreciate that elements of the aspects of the MSCs or the pharmaceutical composition for use in the treatment of chronic kidney disease, or of the MSCs or pharmaceutical composition for use wherein creatinine levels in felines and canines diagnosed with or suffering from chronic kidney disease are reduced, compared to a feline and canine which has not been treated with said MSCs or composition as described above return in the aspect of the pharmaceutical composition of the invention. Consequently, all aspects of the present invention are related. All features and advantages as described in one of the aspects as described above, can relate to any of these aspects, even if they are described in conjunction with a specific aspect.

The MSCs or the pharmaceutical composition comprising MSCs for use according to the current invention, possibly together with further components as described above, will by preference be frozen in order to allow long-time storage of the MSCs or composition. Preferably the MSCs or composition will be frozen at low and constant temperature, such as a temperature below −20° C. These conditions allow a save storage of the MSCs or composition, and enable the MSCs to keep their biological and morphological characteristics, as well as their high cell viability during storage and once thawed.

In a more preferred embodiment, the MSCs or the pharmaceutical composition comprising MSCs for use of the invention can be stored for at least 6 months at a maximum temperature of −80° C., optionally in liquid nitrogen. A crucial factor in the freezing of the MSCs is a cryogenic medium, in particular comprising DMSO. DMSO prevents ice crystal formation in the medium during the freezing process, but may be toxic to the cells in high concentrations. In a preferred embodiment, the concentration of DMSO comprises up to 20%, more preferably up to 15%, more preferably the concentration of DMSO in the cryogen comprises 10%. The cryogenic medium further comprises low-glucose medium such as low glucose DMEM (Dulbecco's Modified Eagle Medium).

Afterwards, the MSCs or the pharmaceutical composition for use of the invention are preferably thawed before administration at a temperature around room temperature, preferably at a temperature between 20° C. and 37° C., more preferably at a temperature between 25° C. and 37° C., and in a time span of maximal 20 minutes, preferably maximal 10 minutes, more preferably maximal 5 minutes.

Furthermore, the MSCs or composition is preferably administered within 2 minutes after thawing, in order to safeguard the vitality of the MSCs.

The invention is further described by the following non-limiting examples which further illustrate the invention, and are not intended to, nor should they be interpreted to, limit the scope of the invention.

### EXAMPLES

The present invention will now be further exemplified with reference to the following examples. The present invention is in no way limited to the given examples.

### Example 1: Mixed Lymphocyte Reaction (MLR) in Healthy Cats

Set-Up:

In order to investigate the immunomodulatory properties of ePB (equine peripheral blood derived)-MSCs in cats, ten healthy cats are intravenously (IV) injected with a composition comprising 3×10⁵ ePB-MSCs in DMEM low glucose and 10% DMSO, in a volume of 1 ml, according to an embodiment of the invention at three time points (T0, T1 and T2) with 2 weeks in between each injection. The ten healthy cats, 4 males and 6 females, are of different breeds, in particular European shorthair, European longhair and Maine Coon, with a mean age of 6±4 years old.

Isolation and Cultivation of ePB-MSCs

According to previously described methods, the ePB-MSCs are isolated from venous blood collected from the vena jugularis of one donor horse. Prior to cultivation of the ePB-MSCs, serum is tested for the presence of multiple transmittable diseases as described by Broeckx et al. 2012. Subsequently the stem cells are cultivated in a Good manufacturing practice (GMP)-certified production site according to GMP-guidelines until passage (P) 5 and characterized on viability, morphology, presence of cell surface markers and population doubling time. Evaluation of the presence (Cluster of Differentiation CD29, CD44 and CD90) and absence (Major Histocompatibility Complex (MHC) II and CD45) of specific cell surface markers is accomplished by using flow cytometry as previously described (Spaas et al., 2013). However, the detailed expression and secretion pattern has been previously described in WO 2020/182935.

The cell viability is assessed using trypan blue. Afterwards, the cells are further cultivated until P10, trypsinized and resuspended at a final concentration of 300.000 cells/mL in Dulbecco's Modified Eagle Medium (DMEM) low glucose with 10% dimethylsulfoxide (DMSO). The ePB-MSCs are stored at −80° C. in cryovials until further use. Sterility of the final product is tested by the absence of aerobic bacteria, anaerobic bacteria, fungi, endotoxins and Mycoplasma.

Study

All cats are daily inspected by the caretaker and undergo a full physical examination at day 0 (T0), week 2 (T1), week 4 (T2) and week 6 (T3) by a veterinarian consisting of the assessment of rectal temperature, heart rate, respiratory rate, mucosal membranes appearance and capillary refill time, together with a hematological and biochemical analysis.

Furthermore, a modified mixed lymphocyte reaction (MLR) is performed at T0 (before treatment administration) and T3 (two weeks after the last (third) treatment) with fresh peripheral blood mononuclear cells (PBMCs) from each individual cat. This assay investigates the immunomodulatory (via stimulated PBMCs) properties of ePB-MSCs. To stimulate PBMCs, they are co-incubated with concanavalin A (ConA).

At T0, T1 and T2, after general physical examination, cats were intravenously (i.v.) injected with 3×10⁵ ePB-MSCs. After thawing the cryovial in the palm of a hand, the content was checked for transparency and clearness and the cell suspension was immediately injected using a 22 G i.v. catheter.

During the MLR assay, the immunomodulatory properties of the ePB-MSCs are investigated by co-incubating these cells with concanavalin A (ConA) stimulated feline PBMCs for four days and assessing the proliferation of the feline PBMCs. Non-stimulated feline PBMCs or stimulated feline PBMCs are used as negative and positive control, respectively. Consequently, PBMC proliferation (%) is evaluated using flow cytometry using Carboxyfluorescein succinimidyl ester 7-aminoactinomycin D (CFSE-7-AAD) labeling. This assay is performed before and after treatment for all cats.

For this, venous feline blood was collected in EDTA blood collection tubes from each individual cat and diluted with HBSS and layered upon an equal amount of Percoll density gradient. After centrifugation on Percoll, the interphase containing the PBMCs was collected. The PBMCs were washed 3 times. Next, PBMCs from each cat were brought to a concentration of 1×10⁶ cells per mL. Then, the PBMCs were labeled with CFSE using 1 μL of CFSE solution per mL of PBMC cell suspension. The CFSE labeled PBMCs were washed and resuspended in MLR medium (DMEM supplemented with 20% FBS, 1% AB/AM (Antibiotics/Antimycotics) and 1% BME (B-mercaptoethanol) 100×) to a final concentration of 2×10⁶ PBMCs per mL. Then, the ConA-solution was added to all the wells of the plates except for the negative control samples. Finally, PBMCs of the designated cats were added to the associated wells. After 4 days of incubation all samples were transferred to FACS tubes, centrifuged and stained with 7-AAD for flow cytometry analysis.

Results:

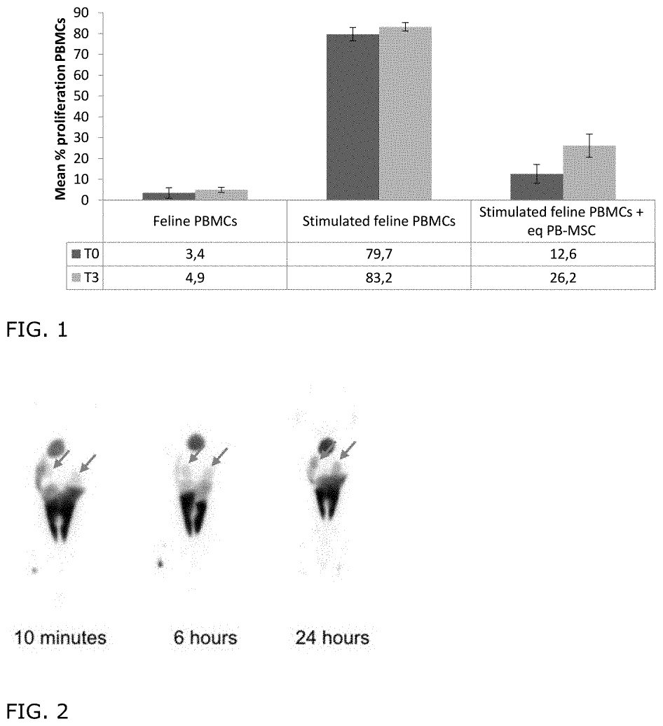

At both timepoints (T0 and T3), the proliferation of the co-cultured ePB-MSCs with stimulated feline PBMCs (T0: 12.6±10%, T3: 26.2±9.8%) is significantly higher compared to the associated negative control (T0: 3.4±2.7%, T3: 4.9±1.3%) (p-value=0.05 and 0.008, respectively). However, the proliferation of the co-culture is significantly lower than the positive control at baseline (79.7±4.7%) (p-value=0.008) and after treatment (83.2±5.7%) (p-value=0.008). No significant difference in mean PBMC proliferation can be found in the co-culture of ePB-MSCs with stimulated feline PBMCs after treatment (26.2±9.8%) compared to baseline (12.6±10%) (p-value=0.017) (FIG. 1).

Conclusion:

The results of current study confirm the immunomodulatory properties of ePB-MSCs on feline PBMCs. This indicates the xenogeneic ePB-MSCs can be used in the treatment of cats.

### Example 2: Mixed Lymphocyte Reaction (MLR) in Healthy Dogs Before and After Treatment with ePB-MSCs

Set-Up:

In order to investigate the immunomodulatory properties of ePB (equine peripheral blood derived)-MSCs in dogs, twelve healthy dogs are intravenously (IV) injected with a composition comprising 3×10⁵ ePB-MSCs in DMEM low glucose and 10% DMSO, in a volume of 1 ml, according to an embodiment of the invention at three time points (T0, T1 and T2) with 2 weeks in between each injection.

Isolation and cultivation of ePB-MSCs is performed as described in Example 1 above.

Study

All dogs are daily inspected by the caretaker and undergo a full physical examination at day 0 (T0), week 2 (T1), week 4 (T2) and week 6 (T3) by a veterinarian consisting of the assessment of rectal temperature, heart rate, respiratory rate, mucosal membranes appearance and capillary refill time, together with a hematological and biochemical analysis.

Furthermore, a modified mixed lymphocyte reaction (MLR) is performed at T0 (before treatment administration) and T3 (two weeks after the last (third) treatment) with fresh peripheral blood mononuclear cells (PBMCs) from each individual dog. This assay investigates the immunomodulatory (via stimulated PBMCs) properties of ePB-MSCs. To stimulate PBMCs, they are co-incubated with concanavalin A (ConA).

At T0, T1 and T2, after general physical examination, cats were intravenously (i.v.) injected with 3×10⁵ ePB-MSCs. After thawing the cryovial in the palm of a hand, the content was checked for transparency and clearness and the cell suspension was immediately injected using a 22 G i.v. catheter.

During the MLR assay, the immunomodulatory properties of the ePB-MSCs are investigated by co-incubating these cells with concanavalin A (ConA) stimulated canine PBMCs for four days and assessing the proliferation of the canine PBMCs. Non-stimulated canine PBMCs or stimulated canine PBMCs are used as negative and positive control, respectively. Consequently, PBMC proliferation (%) is evaluated using flow cytometry using Carboxyfluorescein succinimidyl ester 7-aminoactinomycin D (CFSE-7-AAD) labeling. This assay is performed before and after treatment for all dogs.

For this, venous canine blood was collected in EDTA blood collection tubes from each individual dog and diluted with HBSS and layered upon an equal amount of Percoll density gradient. After centrifugation on Percoll, the interphase containing the PBMCs was collected. The PBMCs were washed 3 times. Next, PBMCs from each dog were brought to a concentration of 1×10⁶ cells per mL. Then, the PBMCs were labeled with CFSE using 1 μL of CFSE solution per mL of PBMC cell suspension. The CFSE labeled PBMCs were washed and resuspended in MLR medium (DMEM supplemented with 20% FBS, 1% AB/AM (Antibiotics/Antimycotics) and 1% BME (B-mercaptoethanol) 100×) to a final concentration of 2×10⁶ PBMCs per mL. Then, the ConA-solution was added to all the wells of the plates except for the negative control samples. Finally, PBMCs of the designated dogs were added to the associated wells. After 4 days of incubation all samples were transferred to FACS tubes, centrifuged and stained with 7-AAD for flow cytometry analysis.

Results:

At both timepoints (T0 and T3), the proliferation of the co-cultured ePB-MSCs with stimulated canine PBMCs is significantly higher compared to the associated negative control. However, the proliferation of the co-culture is significantly lower than the positive control at baseline and after treatment. No significant difference in mean PBMC proliferation can be found in the co-culture of ePB-MSCs with stimulated canine PBMCs after treatment compared to baseline.

Conclusion:

The results of current study confirm the immunomodulatory properties of ePB-MSCs on canine PBMCs. This indicates the xenogeneic ePB-MSCs can be used in the treatment of dogs.

### Example 3: Safety and Efficacy of ePB-MSCs in Feline Chronic Kidney Disease

Set-Up:

A 13-year-old cat suffering from chronic kidney disease (CKD) is intravenously (IV) injected with 3×10⁵ ePB-MSCs of the invention in DMEM low glycose and 10% DMSO, 1 ml. The cat is daily inspected by the caretaker and undergoes a full physical examination at day 0, day 7, day 14, day 21 and month 3 by a veterinarian. The veterinarian contacts the caretaker of the cat by telephone 4 months after the study start to follow-up on the animal. At day 0, an IRIS (International Renal Interest Society) stage (see Table 1) is determined based on the blood creatinine concentration (ranging from “at risk” to stage 4 which is the end stage).

TABLE 1
IRIS stages of chronic kidney disease in cats and dogs

| | Blood creatinine (mg/dl) | | |
|---|---|---|---|
| Stage | Cats | Dogs | |
| 1 | <1.6 | <1.4 | Normal blood creatinine. Some other renal abnormality |
| | | | present (such as inadequate urinary concentrating |
| | | | ability without identifiable non-renal, abnormal renal |
| | | | palpation or renal imaging findings, proteinuria of renal |
| | | | origin, abnormal renal biopsy results, increasing blood |
| | | | creatinine concentrations in samples collected serially). |
| 2 | 1.6-2.8 | 1.4-2.8 | Normal or mildly increased creatinine, mild renal |
| | | | azotemia (lower end of the range lies within reference |
| | | | ranges for creatinine for many laboratories, but the |
| | | | insensitivity of creatinine concentration as a screening |
| | | | test means that patients with creatinine values close to |
| | | | the upper reference limit often have excretory failure). |
| 3 | 2.9-5.0 | 2.9-5.0 | Moderate renal azotemia. Many extra-renal signs may |
| | | | be present, but their extent and severity may vary. If |
| | | | signs are absent, the case could be considered as early |
| | | | Stage 3, while presence of many or marked systemic |
| | | | signs might justify classification as late Stage 3. |
| 4 | >5.0 | >5.0 | Increasing risk of systemic clinical signs and uremic |
| | | | crises. |

Results:

An IRIS stage of 2 is determined for the cat at day 0. A reduction of blood creatinine concentration is seen in the cat after 3 months of the study resulting in an IRIS stage 1. 4 months after the first injection, the cat has an improvement of its quality of life compared to before the study and has gained 10% of body weight.

Conclusion:

The clinical case study demonstrates the intravenous injection of cats with ePB-MSCs results in a significant improvement of quality of life score and body weight, indicating ePB-MSCs is a promising solution for the treatment of chronic kidney disease in cats.

### Example 4: Safety and Efficacy of ePB-MSCs in Canine Chronic Kidney Disease

Set-Up:

A dog suffering from chronic kidney disease (CKD) is intravenously (IV) injected with 3×10⁵ ePB-MSCs of the invention in DMEM low glycose and 10% DMSO, 1 ml. The dog is daily inspected by the caretaker and undergoes a full physical examination at day 0, day 7, day 14, day 21 and month 3 by a veterinarian. The veterinarian contacts the caretaker of the dog by telephone 4 months after the study start to follow-up on the animal. At day 0, an IRIS stage is determined based on the blood creatinine concentration (ranging from “at risk” to stage 4 which is the end stage).

Results:

An IRIS stage of 2 is determined for the dog at day 0. A reduction of blood creatinine concentration is seen in the dog after 3 months of the study resulting in an IRIS stage 1. 4 months after the first injection, the dog has an improvement of its quality of life compared to before the study and has gained body weight.

Conclusion:

The clinical case study demonstrates the intravenous injection of dogs with ePB-MSCs results in a significant improvement of quality of life score and body weight, indicating ePB-MSCs is a promising solution for the treatment of chronic kidney disease in dogs.

### Example 5: Biodistribution of ePB-MSCs in Healthy Cats

Set-Up:

This is a pilot study to evaluate the biodistribution of equine peripheral blood-derived mesenchymal stem cells (ePB-MSCs) following an intravenous (IV) injection in healthy cats. Three cats (at least 10 months old, 2 males and 1 female) are injected with ^{99m}Tc labelled ePB-MSCs (in DMEM low glucose medium), further referred to in this example as the investigational veterinary product (the IVP), and a control product (CP, DMEM low glucose medium). The three healthy cats are injected intravenously (T1: day 0 and T3: day 14) with both a CP (freshly eluted ^{99m}Tc dissolved in DMEM, T1) and the ^{99m}Tc-labelled ePB-MSCs (T3). The distribution of the CP and the ePB-MSCs is assessed subjectively through the whole body using a two-headed gamma camera. The start of the first acquisition is within one hour following the injection of the radioactive compound. Next, one total body scan is performed 6 h and 24 h after placebo control and labelled ePB-MSCs administration.

Results:

All three cats are treated intravenously with the IVP (=ePB-MSCs) and controlled for adverse events. No serious adverse events, no suspected adverse drug reactions and no abnormal clinical signs are observed in the animals after injection with the IVP.

Intravenous injection of the CP leads to an accumulation of free 99mTc in the heart, lung, stomach, bladder, thyroid and salivary glands. In one cat, an increased uptake in the liver is also observed and for another cat radioactive accumulation is measured in the intestines. The highest uptake was seen in the stomach.

Intravenous injection of the IVP (ePB-MSCs) leads to increased radiopharmaceutical uptake in the lung, liver, kidneys and bladder (FIG. 2, arrows mark the location of the kidneys).

Conclusion:

Biodistribution of the ePB-MSCs following IV injection is mainly observed in the lung and present in the liver and kidneys. These results address the natural homing behavior of the ePB-MSCs to the kidneys of cats after intravenous injection and further support their use in treatment for feline chronic kidney disease.

### Example 6: Biodistribution of ePB-MSCs in Healthy Dogs

Set-Up:

This is a pilot study to evaluate the biodistribution of equine peripheral blood-derived mesenchymal stem cells (ePB-MSCs) following an intravenous (IV) injection in healthy dogs. Four dogs (at least 10 months old, 2 males and 2 female) are injected with ^{99m}Tc labelled ePB-MSCs (in DMEM low glucose medium and 10% DMSO), further referred to in this example as the investigational veterinary product (the IVP), and a control product (CP, DMEM low glucose medium and 10% DMSO). The four healthy dogs are injected intravenously (T1: day 0 and T2: day 7) with both a CP (freshly eluted ^{99m}Tc dissolved in DMEM, T1) and the ^{99m}Tc-labelled ePB-MSCs (T2). The distribution of the CP and the ePB-MSCs is assessed throughout the whole body using a two-headed gamma camera. The start of the first acquisition is within one hour following the injection of the radioactive compound. Next, total body scans are performed 10 min, 20 min, 30 min, 40 min, 50 min, 60 min, 3 h, 4 h, 6 h, 8 h, 12 h and 24 h after both placebo control and labelled ePB-MSCs administration. An additional scan is performed 36 h after labelled ePB-MSCs administration.

Results:

All four dogs are treated intravenously with the IVP (=ePB-MSCs) and evaluated for adverse events. No serious adverse events, no suspected adverse drug reactions and no abnormal clinical signs are observed in the animals after injection with the IVP or CP. Intravenous injection of the IVP (ePB-MSCs) leads to a predominant increased radiopharmaceutical uptake in the liver. Furthermore, increased radiopharmaceutical uptake was observed in the heart, lung and bladder, and in addition a minor increased radiopharmaceutical uptake was observed in the kidneys and spleen.

Intravenous injection of the CP leads to an accumulation of free 99mTc in the heart, lung, liver, stomach, bladder, thyroid and salivary glands.

Conclusion:

Biodistribution of the ePB-MSCs following IV injection is mainly observed in the liver and also present in the heart, lung, spleen and kidneys. These results address the natural homing behavior of the ePB-MSCs to the kidneys of dogs after intravenous injection and further support their use in treatment for chronic kidney disease.

### Example 7: Evaluation of Efficacy and Safety of ePB-MSCs in Cats Suffering from CKD

Set-Up:

The safety and efficacy of equine peripheral blood-derived mesenchymal stem cells (ePB-MSCs) was evaluated following an intravenous (IV) injection in cats suffering from chronic kidney disease stage 2 or 3. Four cats were treated with ePB-MSCs (in DMEM low glucose medium supplemented with 10% DMSO), further referred to in this example as the investigational veterinary product (the IVP), and two cats were treated with placebo (Vetivex 9 mg/mL). All cats were injected intravenously, independent of the type of treatment (ePB-MSCs versus Vetivex). During the follow-up period of 12 weeks, a hematological and serum biochemistry analysis, urine analysis, general clinical assessment, quality of life assessment and injection site observation were performed. Quality of life was assessed based on the Quality of Life questionnaire derived from the validated questionnaire as reported by Bijsmans et al. (Bijsmans E S, Jepson R E, Syme H M, Elliott J, Niessen S J. Psychometric Validation of a General Health Quality of Life Tool for Cats Used to Compare Healthy Cats and Cats with Chronic Kidney Disease. J Vet Intern Med. 2016 January-February; 30(1):183-91. doi: 10.1111/jvim.13656. Epub 2015 Nov. 14. PMID: 26567089; PMCID: PMC4913638.). An important parameter that needs to be taken into account is the creatinine concentration in the blood, since CKD is staged based on this creatinine concentration (a higher CKD stage is defined by a higher creatinine concentration in the blood).

Results:

Four cats were treated intravenously with the IVP (=ePB-MSCs) and evaluated for adverse events. No serious adverse events related to the IVP or to the placebo, no suspected adverse drug reactions and no abnormal clinical signs were observed in the animals after injection with the IVP or placebo. The IV injection of the IVP (=ePB-MSCs) led to an increased quality of life (Table 3). Furthermore, the cats treated with the placebo (=Vetivex) showed an increase in creatinine values, while the cats treated with the IVP (=ePB-MSCs) showed a slight decrease in creatinine concentration, indicating a better progression (Table 2). The mean body weight of the cats treated with the IVP (=ePB-MSCs) remained stable (Table 4).

TABLE 2
Mean creatinine values per group (μmol/l)
Creatinine values (μmol/l)

| | ePB-MSCs | placebo |
|---|---|---|
| Day 0 | 208 | 221 |
| Week 12 | 196 | 241 |

TABLE 3
Mean Quality of life per group (score)
Quality of Life (score)

| | ePB-MSCs | placebo |
|---|---|---|
| Day 0 | 3 | −7 |
| Week 12 | 10 | −4 |

TABLE 4
Mean body weight per group (kg)
Body weight (kg)

| | ePB-MSCs | placebo |
|---|---|---|
| Day 0 | 4.6 | 4.4 |
| Week 12 | 4.6 | 4.3 |

Conclusion:

Cats suffering from CKD stage 2 or 3 treated with ePB-MSCs showed an improvement in the quality of life of the animal. Additionally, the creatinine values in these treated cats show a slight decrease in contrast to the placebo treated cats. These results show initial efficacy data of the ePB-MSCs in cats suffering from CKD stage 2 or 3 after intravenous injection and further support their use in the treatment of chronic kidney disease.

The present invention is in no way limited to the embodiments described in the examples and/or shown in the figures. On the contrary, methods according to the present invention may be realized in many different ways without departing from the scope of the invention.

1. Mesenchymal stem cells (MSCs) or a pharmaceutical composition comprising a therapeutically effective amount of MSCs for use in the treatment of chronic kidney disease in felines and canines.

2. The MSCs or the pharmaceutical composition comprising the therapeutically effective amount of MSCs for use according to claim 1, wherein said MSCs are intravenously administered.

3. The MSCs or the pharmaceutical composition comprising the therapeutically effective amount of MSCs for use according to claim 1, wherein said MSCs are native.

4. The MSCs or the pharmaceutical composition comprising the therapeutically effective amount of MSCs for use according to claim 1, wherein said MSCs are derived from blood.

5. The MSCs or the pharmaceutical composition comprising the therapeutically effective amount of MSCs for use according to claim 1, wherein said MSCs are allogeneic or xenogeneic MSCs.

6. The MSCs or the pharmaceutical composition comprising the therapeutically effective amount of MSCs for use according to claim 1, wherein said MSCs are animal-derived.

7. The MSCs or the pharmaceutical composition comprising the therapeutically effective amount of MSCs for use according to claim 1, wherein a dose of 10⁵-10⁷ MSCs per feline and canine is administered.

8. The MSCs or the pharmaceutical composition comprising the therapeutically effective amount of MSCs for use according to claim 1, wherein a single dose is administered.

9. The MSCs or the pharmaceutical composition comprising the therapeutically effective amount of MSCs for use according to claim 1, wherein multiple doses are administered with each dose being administered at different time points.

10. The MSCs or the pharmaceutical composition comprising the therapeutically effective amount of MSCs for use according to claim 1, wherein one dosage of said composition has a volume of maximally about 5 ml.

11. The MSCs or the pharmaceutical composition comprising the therapeutically effective amount of MSCs for use according to claim 1, wherein said MSCs measure negative for MHC class II molecules and/or CD45.

12. The MSCs or the pharmaceutical composition comprising the therapeutically effective amount of MSCs for use according to claim 1, wherein said MSCs measure positive for mesenchymal markers CD29, CD44 and CD90 and measure negative for MHC class II molecules and CD45.

13. The MSCs or the pharmaceutical composition comprising the therapeutically effective amount of MSCs for use according to claim 1, wherein said MSCs secrete immunomodulatory prostaglandin E2 cytokine when present in an inflammatory environment or condition.

14. The MSCs or the pharmaceutical composition comprising the therapeutically effective amount of MSCs for use according to claim 1, wherein said MSCs have an increased secretion of at least one of the molecules chosen of IL-6, IL-10, TGF-β, NO, or a combination thereof; and/or a decreased secretion of IL-1 when present in an inflammatory environment or condition and compared to a cell having the same characteristics but not being subjected to said inflammatory environment or condition.

15. The MSCs or the pharmaceutical composition comprising the therapeutically effective amount of MSCs for use according to claim 1, wherein said MSCs stimulate the expression of PgE2, IL-6, IL-10, NO, or a combination thereof when in the presence of PBMCs and/or suppress the secretion of TNF-α, IFN-γ, IL-1, TGF-β, IL-13 or a combination thereof when in the presence of PBMCs.

16. The MSCs or the pharmaceutical composition comprising the therapeutically effective amount of MSCs for use according to claim 1, wherein said MSCs are present in a sterile liquid.

17. The MSCs or the pharmaceutical composition comprising the therapeutically effective amount of MSCs for use according to claim 1, wherein creatinine levels in felines and canines diagnosed with or suffering from chronic kidney disease are reduced, compared to a feline or canine which has not been treated with said MSCs or composition.

18. The MSCs or the pharmaceutical composition comprising the therapeutically effective amount of MSCs according to claim 17, wherein said MSCs or pharmaceutical composition are intravenously administered.

19. A pharmaceutical composition comprising peripheral blood-derived MSCs, said MSCs are animal-derived and present in a sterile liquid at a concentration of between 10⁵-10⁷ MSCs per mL of said composition, wherein said composition has a volume of about 0.5 to 5 ml, wherein said MSCs measure positive for mesenchymal markers CD29, CD44 and CD90 and measure negative for MHC class II molecules and CD45, and wherein said MSCs have a suspension diameter between 10 μm and 100 μm.
